(19) United States
(12) Patent Application Publication
(10) Pub. No.: US 2003/0060499 A1
(43) Pub. Date: Mar. 27, 2003

(54) Novel therapeutic method

(76) Inventor: Ian Frederic Tulloch, Sawston (GB)

Correspondence Address: GLAXOSMITHKLINE, Corporate Intellectual Property UW2220, P.O. Box 1539, King of Prussia, PA, 19406-0939, US

(21) Appl. No.: 10/208,072

(22) Filed: Jul. 29, 2002

Related U.S. Application Data
(63) Continuation of application No. 09/929,758, filed on Aug. 14, 2001, now abandoned.
Application No. 09/929,758 is a continuation of application No. 09/324,060, filed on Jun. 1, 1999, now abandoned.
(60) Provisional application No. 60/089,691, filed on Jun. 18, 1998.

(51) Int. Cl. A61K 31/405

(52) U.S. Cl. 514/421

(57) ABSTRACT

A therapeutic method for preserving the dopaminergic function of patients suffering from Parkinson's disease, which method comprises administering an effective amount of ropinirole or a pharmaceutically acceptable salt or solvate thereof to a human or animal patient in need thereof. Typically, said patient has had Parkinson's disease for a period of less than three years since diagnosis.

Preferably the invention comprises administering to said patient an effective amount of ropinirole or a pharmaceutically acceptable salt or solvate thereof, optionally in combination with one or more other dopamine agonists, in the absence of levodopa or any other dopamine precursor, and thereafter treating the patient with levodopa.

### FIELD OF THE INVENTION

The present invention relates to a novel therapeutic method for treating patients suffering from Parkinson's disease. In particular, the present invention seeks to provide a method of preserving the dopaminergic function of presynaptic dopamine neurones in parkinsonian patients.

### BACKGROUND TO THE INVENTION

Parkinson's disease, which is estimated to effect between 500,000 and 1,000,000 Americans, is a chronic and progressive disorder that results from the death of nerve cells in a critical area of the brain called the substantia nigra. These nerve cells normally produce dopamine, a chemical messenger that plays an important role in motor movement control by transmitting signals between the substantia nigra and another critical area of the brain called the striatum. Dopamine depletion, for example as a result of the death of the dopamine neurones, impairs a patient's ability to control motor movements. By the time a patient with Parkinson's disease presents, he or she has normally already lost an estimated 80% of his or her dopamine neurones. At this stage, the remaining dopamine nerve cells of the substantia nigra are unable to produce enough dopamine to maintain effective motor control, and the symptoms of Parkinson's disease are apparent.

At the time of writing a number of therapeutic methods are known and practised in the art for treating the symptoms of Parkinson's disease. The most common symptomatic treatment of Parkinson's involves the administration of levodopa, which is a natural precursor of dopamine. Levodopa is widely thought to mediate neurotoxic effects however, possibly through the oxidation of excess dopamine to form free-radicals, and this may exacerbate the decline in the dopamine neurone population of a patient. Prolonged treatment with levodopa has also been associated with dyskinesias. Moreover, it is well known that after a treatment period of 5 to 10 years parkinsonian patients generally become progressively less responsive to treatment with levodopa, perhaps as a result of the ongoing death of the dopamine neurones which are required to uptake the administered levodopa and convert it to dopamine. As the disease progresses therefore, greater doses of levodopa are needed to maintain effective motor control. As the daily dose of levodopa is increased, the neurotoxic effects that result from use of the drug may become more pronounced. Eventually, the patient will become substantially unresponsive to treatment with levodopa. Levodopa does not appear to halt or delay the disappearance of dopamine producing cells, and it would therefore be desirable to defer treatment with levodopa for as long as possible.

Other treatments for Parkinson's comprehend the administration of an effective amount of a dopamine agonist such, for example, as bromocriptine or pergolide which acts at the D₂ receptors of the basal ganglia to provide motor control. It is widely believed that dopamine agonists are generally not as effective in treating the symptoms of Parkinson's disease as levodopa, and as a result they are most commonly employed as second and/or third line treatments for Parkinson's, with levodopa being the corresponding first line of treatment.

Another D₂-type dopamine agonist that is approved for use in the symptomatic treatment of Parkinson's disease is 4-[2-(di-n-propylamino)ethyl]-1,3-dihydro-2H-indolin-2-one hydrochlorid (ropinirole hydrochloride). Ropinirole was originally developed as an antihypertensive and anti-anginal agent as described in U.S. Pat. No. 4,452,808 and U.S. Pat. No. 4,588,740). Subsequently, ropinirole was found to be a potent CNS active, non-ergot dopamine receptor agonist (see U.S. Pat. No. 4,824,860 and U.S. Pat. No. 4,912,126) at the D₂, D₃ and D₄ receptors of the basal ganglia.

Processes for the production of ropinirole hydrochloride are disclosed in U.S. Pat. No. 4,997,954 and U.S. Pat. No. 5,336,781.

### OBJECTS OF THE INVENTION

An object of the present invention is to provide an improved method of treating Parkinson's disease.

Another object of the present invention is to provide a method of preserving the dopaminergic function of dopamine neurones in parkinsonian patients.

Another object of the present invention is to provide a novel combination therapy for the treatment of Parkinson's disease by which the administration of levodopa can be deferred for as long as possible.

It has now been surprisingly discovered that the administration of ropinirole appears to slow the decline of the dopamine neurone population, and thus preserve the dopaminergic function of the substantia nigra, in parkinsonian patients. In particular, through the use of 18-Fluoro-dopa Positron Emission Tomography (PET), it has been found that ropinirole administered to patients suffering from Parkinson's disease, especially those patients that have had the disease for three years or less since diagnosis, appears to act presynaptically to preserve the dopaminergic function of the dopamine nerve cells.

### SUMMARY OF THE INVENTION

Accordingly, the present invention provides a therapeutic method for preserving the dopaminergic function of patients suffering from Parkinson's disease, which method comprises administering an effective amount of ropinirole or a pharmaceutically acceptable salt or solvate thereof to a human or animal patient in need thereof.

The therapeutic method of the present invention may preferably comprise administering ropinirole to a patient who has had Parkinson's disease for a period of less than three years since diagnosis, most preferably less than two years. It has been found that in patients with Parkinson's disease for less than two years, there appears to be a slower loss of dopaminergic function for patients treated with ropinirole as compared with those patients treated with levodopa.

In some embodiments, the treatment method of the present invention may comprise administering to a parkinsonian patient who has had the disease for less than three years, an effective amount of ropinirole or a pharmaceutically acceptable salt or solvate thereof as a first line therapy, optionally in combination with one or more other dopamine agonists, in the absence of levodopa or any other dopamine precursor, and thereafter treating the patient with levodopa. Treatment with levodopa may begin when the treatment with ropinirole becomes significantly less effective than when treatment was started. Preferably said treatment with ropinirole may be commenced as soon as Parkinson's disease is diagnosed and may be continued for a period of 1 to 10 years, preferably 2 to 5 years until the symptomatic relief provided by ropinirole has declined to an unacceptable level; thereafter treatment with levodopa may be started, optionally in conduction with ropinirole. Said second line treatment with levodopa may be continued until the patient fails to respond adequately to said second line treatment, which may be after a period of 1 to 10 years, typically 2 to 5 years.

The present invention also provides the use of ropinirole or a pharmaceutically acceptable salt or solvate thereof in the manufacture of a medicament for preserving the dopaminergic function of dopamine neurones of patients suffering from Parkinson's disease.

### DESCRIPTION OF THE INVENTION

Ropinirole used in the present invention is suitably in the form of the free base or a pharmaceutically acceptable salt thereof. A preferred pharmaceutically acceptable salt of ropinirole is crystalline hydrochloride. Suitable procedures for preparing ropinirole hydrochloride include those mentioned in U.S. Pat. No. 4,997,954, and preferably those mentioned in U.S. Pat. No. 5,336,781.

A medicament for use in preserving dopaminergic function of dopamine nerve cells of patients suffering from Parkinson's disease may be prepared by a mixture of ropinirole or a pharmaceutically acceptable salt or solvate thereof with an appropriate carrier, which may contain a diluent, binder, filler, disintegrant, flavouring agent, colouring agent, lubricant or preservative in conventional manner.

Preferably, the medicament is in unit dosage form and in a form adapted for use in the medical or veterinarial fields. For example, such preparations may be in a pack form accompanied by written or printed instructions for use in preserving dopaminergic function in parkinsonian patients.

The suitable dosage range for ropinirole or a pharmaceutically acceptable salt or solvate depends on the relation of potency to absorbability and the frequency and route of administration. Usually an appropriate regimen will be determined for a patient by titration in the usual way for D₂ agonists.

Ropinirole or a pharmaceutically acceptable salt or solvate thereof may be formulated for administration by any route, and examples are oral, sub-lingual, rectal, topical, parenteral, intravenous or intramuscular administration. Preparations may, if desired, be designed to give slow release of the ropinirole or a pharmaceutically acceptable salt or solvate thereof.

The medicaments may, for example, be in the form of tablets, capsules, sachets, vials, powders, granules, lozenges, reconstitutable powders, or liquid preparations, for example solutions or suspensions, or suppositories.

The medicaments, for example those suitable for oral administration, may contain conventional excipients such as binding agents, for example syrup, acacia, gelatin, sorbitol, tragacanth, or polyvinylpyrrolidone; fillers, for example lactose, sugar, maize-starch, calcium phosphate, sorbitol or glycerine; tabletting lubricants, for example magnesium stearate; disintegrants, for example starch, polyvinylpyrrolidone, sodium starch glycollate or microcrystalline cellulose; or pharmaceutically acceptable setting agents such as sodium lauryl sulphate.

Solid medicaments may be obtained by conventional methods of blending, filling, tabletting or the like. Repeated blending operations may be used to distribute ropinirole or a salt or solvate thereof throughout those medicaments employing large quantities of fillers. When the medicament is in the form of a tablet, powder, or lozenge, any carrier suitable for formulating solid pharmaceutical compositions may be used, examples being magnesium stearate, starch, glucose, lactose, sucrose, rice flour and chalk. Tablets may be coated according to methods well known in normal pharmaceutical practice, in particular with an enteric coating. The medicament may also be in the form of an ingestible capsule, for example of gelatin containing ropinirole or a salt thereof if desired with a carrier or other excipients.

Medicaments for oral administration as liquids may be in the form of, for example, emulsions, syrups, or elixirs, or may be presented as a dry product for reconstitution with water or other suitable vehicle before use. Such liquid medicaments may contain conventional additives such as suspending agents, for example sorbitol, syrup, methyl cellulose, gelatin, hydroxyethylcellulose, carboxymethylcellulose, aluminium stearate gel, hydrogenated edible fats; emulsifying agents, for example lecithin, sorbitan monooleate, or acacia; aqueous or non-aqueous vehicles, which include edible oils, for example almond oil, fractionated coconut oil, oily esters, for example esters of glycerine, or propylene glycol, or ethyl alcohol, glycerine, water or normal saline; preservatives, for example methyl or propyl p-hydroxybenzoate or sorbic acid; and if desired conventional flavouring or colouring agents.

Ropinirole or a pharmaceutically acceptable salt or solvate thereof may also be administered by a non-oral route. In accordance with routine pharmaceutical procedure, the medicaments may be formulated, for example for rectal administration as a suppository. They may also be formulated for presentation in an injectable form in an aqueous or non-aqueous solution, suspension or emulsion in a pharmaceutically acceptable liquid, e.g. sterile pyrogen-free water or a parenterally acceptable oil or a mixture of liquids. The liquid may contain bacteriostatic agents, anti-oxidants or other preservatives, buffers or solutes to render the solution isotonic with the blood, thickening agents, suspending agents or other pharmaceutically acceptable additives. Such forms will be presented in unit dose form such as ampoules or disposable injection devices or in multi-dose forms such as a bottle from which the appropriate dose may be withdrawn or a solid form or concentrate which can be used to prepare an injectable formulation.

As mentioned hereinbefore, the effective dose of the ropinirole or pharmaceutically acceptable salt or solvate depends on the condition of the parkinsonian patient and on the frequency and route of administration. Preferably, the composition is in unit dose form. Each dosage unit for oral administration contains preferably from 1 to 50 mg (and for parenteral administration contains preferably from 0.1 to 15 mg) of ropinirole.

The daily dosage required for an adult patient may be, for example, an oral dose of between 1 mg and 100 mg, preferably between 1 mg and 50 mg, more preferably 125 mg; or an intravenous, subcutaneaus or intramuscular dose of between 0.1 mg and 50 mg, preferably between 0.1 mg and 15 mg, of ropinirole, the compound being administered 1 to 4 times per day. Suitably the compound will be administered for a period of continuous therapy.

The daily dosage of ropinirole may be increased progressively throughout the period of treatment. For example, a patient may be given 1-5 mg/day ropinirole at the start of a treatment programme, and this may be increased to 10-25 mg over a period of 6 months to five years or more. The daily dosage may be increased uniformly or non-uniformly throughout this period. In a typical example, a patient may be given 3-4 mg/day ropinirole by an oral route at the commencent of a therapy; this may be increased aggressively to about 10 mg/day over the first 6 months; and thereafter the daily dosage may be increased more slowly to about 15-16 mg/day over the following 4 to 4½ years.

Preferably the present invention is practised using a controlled release or delayed release formulation containing ropinirole or a pharmaceutically acceptable salt thereof.

By controlled release is meant any formulation technique wherein release of the active substance from the dosage from is modified to occur at a slower rater than that from an immediate release product, such as a conventional swallow tablet or capsule.

By delayed release is meant any formulation technique wherein release of the active substance from the dosage form is modified to occur at a later time than that from a conventional immediate release product. The subsequent release of active substance from a delayed release formulation may also be controlled as defined above.

Examples of controlled release formulations which are suitable for incorporating ropinirole are described in:

Sustained Release Medications, Chemical Technology Review No. 177. Ed. J. C. Johnson. Noyes Data Corporation 1980.

Controlled Drug Delivery, Fundamentals and Applications, 2nd Edition. Eds. J. R. Robinson, V. H. L. Lee. Mercel Dekkes Inc. New York 1987.

Examples of delayed release formulations which are suitable for incorporating ropinirole are described in:

Remington's Pharmaceutical Sciences 16th Edition, Mack Publishing Company 1980, Ed. A. Osol.

Such controlled release formulations are preferably formulated in a manner such that release of active substance such as ropinirole is effected predominantly during the passage through the stomach and the small intestine, and delayed release formulations are preferably formulated such that release of active substance such as ropinirole is avoided in the stomach and is effected predominantly during passage through the small intestine

Said formulations are preferably formulated such that the release of the active substance is predominantly 1½ to 3 hours post ingestion.

Preferred formulations are ultimately enteric coated tablets or caplets, wax or polymer coated tablets or caplets or time-release matrices, or combinations thereof.

Thus, a particular aspect of the invention involves use of a polymeric controlled release composition comprising a reaction complex formed by the interaction of (1) a calcium polycarbophil component which is a water-swellable, but water insoluble, fibrous cross-linked carboxy-functional polymer, said polymer containing (a) a plurality of repeating units of which at least about 80% contain at least one carboxyl functionality, and (b) about 0.05 to about 1.5% cross-linking agent substantially free from polyalkenyl polyether, said percentages being based upon the weights of unpolymerised repeating unit and cross-linking agent, respectively, with (2) water, in the presence of ropinirole. The amount of calcium polycarbophil present is from about 0.1 to about 99% by weight, for example about 10%. The amount of active agent present is from about 0.0001 to about 65% by weight, for example between about 5 and 20%: The amount of water present is from about 5 to about 200% by weight, for example between about 5 and 10%. The interaction is carried out at a pH of between about 3 and about 10, for example about 6 to 7. The calcium polycarbophil is originally present in the form of a calcium salt containing from about 5 to about 25% calcium.

Thus, a further particular aspect involves use of a system for the controlled release of ropinirole, comprising (a) a deposit-core comprising an effective amount of ropinirole and having defined geometric form, and (b) a support-platform applied to said deposit-core, wherein said deposit-core contains ropinirole, and at least one member selected from the group consisting of (1) a polymeric material which swells on contact with water or aqueous liquids and a gellable polymeric material wherein the ratio of the said swellable polymeric material to said gellable polymeric material is in the range 1:9 to 9:1, and (2) a single polymeric material having both swelling and gelling properties, and wherein the support-platform is an elastic support, applied to said deposit-core so that it partially covers the surface of the deposit-core and follows changes due to hydration of the deposit-core and is slowly soluble and/or slowly gellable in aqueous fluids. The support-platform may comprise polymers such as hydroxypropylmethylcellulose, plasticizers such as a glyceride, binders such as polyvinylpyrrolidone, hydrophilic agents such as lactose and silica, and/or hydrophobic agents such as magnesium stearate and glycerides. The polymer(s) typically make up 30 to 90% by weight of the support-platform, for example about 35 to 40%. Plasticizer may make up at least 2% by weight of the support-platform, for example about 15 to 20%. Binder(s), hydrophilic agent(s) and hydrophobic agent(s) typically total up to about 50% by weight of the support-platform, for example about 40 to 50%.

The present invention further provides a pharmaceutical composition for use a therapeutic treatment to preserve dopaminergic function in parkinsonian patients, which composition comprises an effective amount of ropinirole or a pharmaceutically acceptable salt or solvate thereof and a pharmaceutically acceptable carrier. Such compositions may be prepared in the manner as hereinbefore described.

### EXAMPLE 1

A study was designed and carried out as follows:

Study Population

One hundred and twenty patients with early Parkinson's disease, and no previous exposure to 1-dopa or dopamine agonists.

Study Design

A double-blind, multicentre, parallel group, two year study of ropinirole at a flexible dose (0.75-24 mg/day) versus 1-dopa (Sinemet, 50-1,000 mg/day). There was a placebo run-in phase of up to 2 weeks. Over the first 4 week titration period of active medication all patients were titrated to dose levels of 3 mg/day ropinirole or 200 mg/day 1-dopa. There was an optional visit halfway through the titration period.

Starting at the week 4 visit, patients received dose levels of 4 mg/day ropinirole or 300 mg/day 1-dopa, after which dose levels were flexible.

For patients who satisfactorily completed the 4 week titration phase, an initial PET scan took place within the following 2 months. A final PET scan took place at the end of the study at 2 years.

Efficacy was also assessed by means of the UPDRS motor score and CGI Severity of Illness (measured at baseline and every subsequent visit), CGI Global Improvement (assessed at every post-baseline visit up to 12 months) and the need for 1-dopa rescue during the study. No motor score assessment was required at the optional 2 week visit.

Safety Parameters

Ophthalmic examinations including electroretinograms were performed on all patients except those whose medical history and concomitant medication precluded this. The examinations were performed within 2 months of completion of the fixed titration period, then at least 6 months after this initial examination and finally at 2 years to detect possible retinal dysfunction. In addition, at one centre only, electro-oculograms (EOG) were performed as part of the electrophysiological examinations.

Adverse experiences, changes to concomitant medication and vital signs (sitting BP and pulse) were recorded at each visit from the baseline visit. Laboratory evaluations of blood chemistry and haematology were made at screening, Month 12 and Month 24.

### EXAMPLE 2

In an ongoing five year, double-blind, multicentre study, patients with early Pakinson's disease (Hoehn & Yahr I-III) were randomised to receive either ropinirole or levodopa. Of those enrolled, 37 patients were scanned using 18 Fluoro-dopa PET scanning within the first year and then approximately two years later to measure the gradual decline in basal ganglia dopaminergic function. Images from both the better (less deteriorated) and worse (more deteriorated) sides of the brain were analysed; however these results focus on the average of both sides of the brain. The following endpoints were assessed:

1 Mean percent change in putamen dopaminergic function in all patients (Hoehn & Yahr I-III)

2 Mean percent change in putamen dopaminergic function in Parkinson's disease patients with symptoms present for under two years

3 Mean percent change in putamen dopaminergic function in Parkinson's disease patients with symptoms present for more than two years

When considering all patients who entered the study, there appeared to be a slower loss of putamen dopaminergic function for patients treated with ropinirole compared with those treated with levodopa (13% versus 18% respectively). In the subgroup pf patients with Parkinson's disease for less than 2 years, this difference became more obvious, the decrease in putamen dopaminergic function was also less for patients treated with ropinirole (14%) versus levodopa treated patients (28%). There was no statistically significant difference between treatment groups for patients with Parkinson's disease for more than two years.

Overall, patients treated with ropinirole who have had Parkinson's disease for less than two years demonstrated the greatest preservation of putamen dopaminergic function.

### EXAMPLE 3

In a three year, international, multicentre, double-blind comparative clinical trial, Parkinson's patients were randomised to receive either ropinirole (n=168) or bromocriptine (n=167). 33% of patients received selegiline concomitantly with either ropinirole or bromocriptine. Of those enrolled, 214 patients completed the study (approximately 39% of patients treated with ropinirole and 33% of patients treated with bromocriptine withdrew from the study). Efficacy was based on the following:

1 Mean Unified Parkinson's Disease Rating Scale (UPDRS) Activities of Daily Living (ADL) score (Part II) at completion

2 Improvement in UPDRS total motor examination score (Part III)

3 Percentage of patients who were considered responders, defined as those with at least a 30% reduction from baseline in total motor examination score on the UPDRS

4 Average combined ADL and motor score at endpoint

5 The percentage of patients who required supplemental levodopa therapy.

Among patients who completed the study, patients treated with ropinirole demonstrated a statistically significantly mean improvement (p=0.009) in the UPDRS ADL score versus patients treated with bromocriptine. In this same patient population, patients treated with ropinirole also showed a greater mean improvement in UPDRS motor score than patients in the bromocriptine group (31% versus 22%, respectively). At the study's completion, 53% of patients treated with ropinirole were considered responders compared with 42% for the bromocriptine group.

In addition, the average improvement in the combined ADL and motor scores were statistically significantly greater for patients treated with ropinirole (p=0.018) versus bromocriptine-treated patients. Fewer patients treated with ropinirole than expected required supplemental levodopa (34% versus 42% for bromocriptine). There was a very low incidence of dyskinesias for both treatment groups (8% for ropinirole versus 7% for bromocriptine).

What is claimed is:

1 A therapeutic method for preserving the dopaminergic function of patients suffering from Parkinson's disease, which method comprises administering an effective amount of ropinirole or a pharmaceutically acceptable salt or solvate thereof to a human or animal patient in need thereof.

2 The method of claim 1, wherein said patient has had Parkinson's disease for a period of less than three years since diagnosis.

3 The method of claim 1, wherein said patient has had Parkinson's disease for a period of less than two years since diagnosis.

4 The method of claim 2, comprising administering to said patient an effective amount of ropinirole or a pharmaceutically acceptable salt or solvate thereof, optionally in combination with one or more other dopamine agonists, in the absence of levodopa or any other dopamine precursor, and thereafter treating the patient with levodopa.

5 The method of claim 4, wherein treatment with levodopa is begun when the treatment with ropinirole becomes significantly less effective than when treatment was started.

6 The method of claim 4, wherein said treatment with ropinirole is commenced as soon as Parkinson's disease is diagnosed.

7 The method of claim 4, wherein treatment with ropinirole is continued for a period of 1 to 10 years

8 The method of claim 4, wherein treatment with ropinirole is continued for a period of 2 to 5 years.

9 The method of claim 4, wherein treatment with levodopa is started, optionally in conjuction with ropinirole after a period of 1 to 10 years.

10 The method of claim 4, wherein treatment with levodopa is started, optionally in conjuction with ropinirole after a period of 2 to 5 years.

11 Use of ropinirole or a pharmaceutically acceptable salt or solvate thereof in the manufacture of a medicament for preserving the dopaminergic function of dopamine neurones of patients suffering from Parkinson's disease.

12 The method of claim 1, wherein each dosage unit for oral administration contains from 1 to 50 mg of ropinirole.

13 The method of claim 1, wherein each dosage unit for parenteral administration contains from 0.1 to 15 mg of ropinirole.

14 The method of claim 1, wherein the daily dosage of ropinirole required for an adult patient for an oral dose is between 1 mg and 100 mg, or for an intravenous, subcutaneaus or intramuscular dose is between 0.1 mg and 50 mg; the compound being administered 1 to 4 times per day.

15 The method of claim 1, wherein, ropinirole is administered for a period of continuous therapy.

16 A pharmaceutical composition for use a therapeutic treatment to preserve dopaminergic function in parkinsonian patients, which composition comprises an effective amount of ropinirole or a pharmaceutically acceptable salt or solvate thereof and a pharmaceutically acceptable carrier.

17 The method of claim 1, wherein the daily dosage of ropinirole is increased throughout the period of treatment.

18 The method of claim 1, wherein the daily dosage of ropinirole is started at about 1-5 mg/day, and is increased to 10-25 mg/day over a period of 6 months to 5 years.

19 The method of claim 1, wherein the daily dosage of ropinirole is started at 3-4 mg/day, and is increased to 10 mg over 6 months.

20 The method of claim 19, wherein the daily dosage of ropinirole is increased to 15-16 mg over the following period of 4 to 4½ years.
